(19) United States
(12) Patent Application Publication
(10) Pub. No.: US 2022/0185075 A1
(43) Pub. Date: Jun. 16, 2022

(54) CONTROL DEVICE AND STORAGE MEDIUM

(71) Applicant: KABUSHIKI KAISHA TOKAI RIKA DENKI SEISAKUSHO, Aichi (JP)

(72) Inventors: Kenji NARUMI, Aichi (JP); Hiroyasu UEDA, Aichi (JP); Ayano ITO, Aichi (JP)

(73) Assignee: KABUSHIKI KAISHA TOKAI RIKA DENKI SEISAKUSHO, Aichi (JP)

(21) Appl. No.: 17/503,775

(22) Filed: Oct. 18, 2021

(30) Foreign Application Priority Data
Dec. 10, 2020 (JP) 2020-204785

(51) Int. Cl. B60H 3/00; A61L 9/20; A61L 9/14

(52) U.S. Cl. CPC B60H 3/0035; A61L 2209/111; A61L 9/14; A61L 9/20

(57) ABSTRACT

To provide a control device and a storage medium capable of improving air environments of target space while considering the use state thereof.

A control device includes: a control unit configured to perform processing of determining a start of use of target space, and processing of controlling, when the start of use has been determined, a fragrance generation device provided in the target space to supply a fragrance into the target space.

### CROSS REFERENCE TO RELATED APPLICATION(S)

This application is based upon and claims benefit of priority from Japanese Patent Application No. 2020-204785, filed on Dec. 10, 2020, the entire contents of which are incorporated herein by reference.

### BACKGROUND

The present invention relates to a control device and a storage medium.

Recently, there have been considered various devices that, for example, deodorize space in a vehicle interior. For example, Japanese Patent Application Laid-open No. 2011-105257 discloses an air conditioner device for vehicles that uses ozone for deodorization and disinfection.

### SUMMARY

### Technical Problem

However, the deodorization, disinfection, and the like of the vehicle interior space are performed in accordance with user's instruction or under the condition that no passenger is in the vehicle interior, and the use state of target space is considered.

Then, in view of the above-described problem, the present invention aims at providing a new and improved control device and storage medium capable of improving air environments of target space while considering the use state thereof.

### Solution to Problem

To solve the above-described problem, there is provided a control device including a control unit configured to perform processing of determining a start of use of target space, and processing of controlling, when the start of use has been determined, a fragrance generation device provided in the target space to supply a fragrance into the target space.

Moreover, to solve the above-described problem, there is provided a control device including a control unit configured to perform processing of determining an end of use of target space, and processing of executing, when the end of use has been determined, an odor reduction device or a disinfection device provided in the target space.

Moreover, to solve the above-described problem, there is provided a non-transitory computer readable storage medium, controlling a computer to function as a control unit that performs: processing of determining a start of use of target space, and processing of controlling, when the start of use has been determined, a fragrance generation device provided in the target space to supply a fragrance into the target space.

### Advantageous Effects of Invention

As described above, the present invention is able to improve air environments of target space while considering the use state thereof.

### BRIEF DESCRIPTION OF THE DRAWINGS

FIG. 1 is a diagram illustrating a configuration example of a space improvement control system according to an embodiment of the present invention.

FIG. 2 is a flowchart illustrating an example of a flow of a space improvement control according to the embodiment.

### DETAILED DESCRIPTION OF THE EMBODIMENT(S)

Hereinafter, referring to the appended drawings, preferred embodiments of the present invention will be described in detail. It should be noted that, in this specification and the appended drawings, structural elements that have substantially the same function and structure are denoted with the same reference numerals, and repeated explanation thereof is omitted.

### 1. Configuration Example

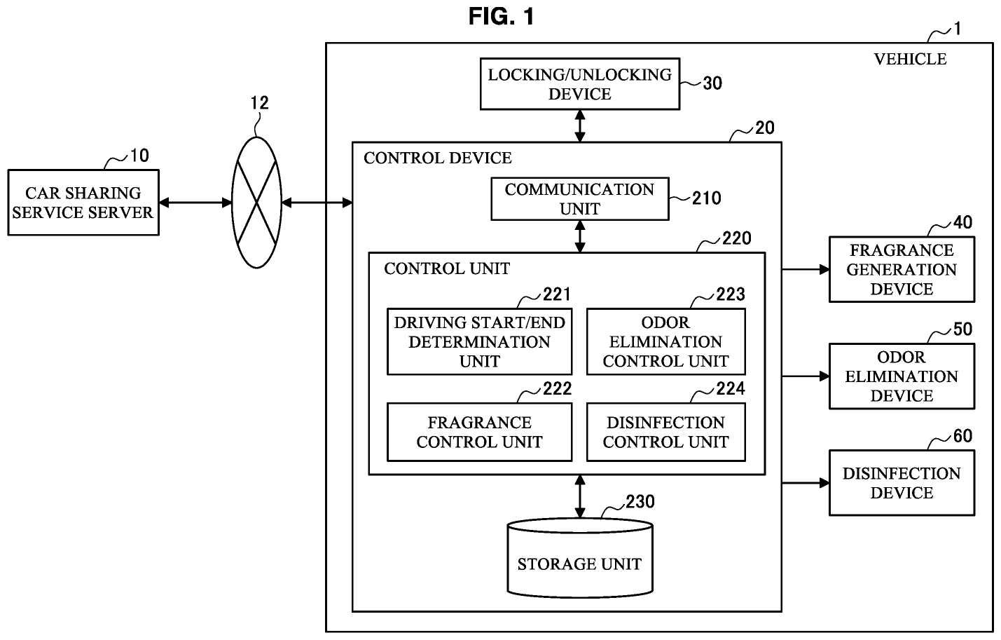

FIG. 1 is a diagram illustrating a configuration of a space improvement control system according to an embodiment of the present invention. The space improvement control system includes a car sharing service server 10 and a control device 20 provided in a vehicle 1, as illustrated in FIG. 1.

The embodiment exemplifies a vehicle interior of the vehicle 1 as target space whose air environments are to be improved. Moreover, the embodiment supposes, as the improvement of air environments, at least any one of addition of a fragrance to space, reduction of an odor in space, and disinfection of viruses and germs in space. The examples of the method of reducing an odor include, for example, odor elimination by removing or alleviating an odor by a chemical action, a biological action, or the like, deodorization by removing or alleviating an odor by a physical action such as decomposition, adsorption, and the like of substances, and deodorization by masking an odor using another fragrance. Moreover, the examples of disinfection achievable by the embodiment include space disinfection for weakening actions of viruses and germs floating in the air and removing them, and object disinfection for weakening actions of viruses and germs attached on objects and removing them. Moreover, the method of disinfection may be sterilization for killing viruses and germs.

Moreover, in the embodiment, the air environments of a vehicle interior can be improved by, for example, at least any one of a fragrance generation device 40, an odor elimination device 50, and a disinfection device 60, which are provided in the vehicle 1.

1-1. Car Sharing Service Server 10

The car sharing service server 10 is an information processing device that performs various controls for car sharing in which a specific vehicle is used commonly among registered members. The car sharing service server 10 controls, for example, member information (a name, age, address, mail address, member ID, and the like), booking information (a member ID, use date/time, getting-on place, vehicle ID, and the like), and vehicle information (a number, vehicle type, vehicle ID, and the like). The car sharing service server 10 is also able to issue an electronic key to a smartphone or the like of a member in cooperation with an authentication server. The issue of such an electronic key (also referred to as an encryption key) is a system for granting only permitted members the right of a key of a specific vehicle only for permitted time. The vehicle is provided with a device corresponding to the electronic key. With an application of a smartphone, a member is able to open and close a door lock of a vehicle or start an engine using the above-described electronic key. In this manner, it is possible to achieve sharing of a vehicle without transfer of a physical key.

1-2. Control Device 20

The control device 20 is provided in the vehicle 1, for example, and communicably connected to the fragrance generation device 40, the odor elimination device 50, and the disinfection device 60 of the vehicle 1 to appropriately perform a control for improving air environments of the vehicle interior of the vehicle 1. Moreover, the use state of the vehicle 1 is obtained on the basis of information acquired from the car sharing service server 10 communicably connected through a network 12 or information acquired from a locking/unlocking device 30 that locks and unlocks the door lock of the vehicle 1. Note that the locking/unlocking device 30 may be a device for locking and unlocking with a physical key, a device for locking and unlocking with an electronic key, or a device for locking and unlocking with any one of a physical key and an electronic key. Moreover, the locking/unlocking device 30 may be a device supporting a so-called smart entry system for unlocking when a given user (concretely, a smartphone, smart key, or the like to which an electronic key is issued) is positioned within a certain distance to the vehicle 1 or when such a user touches the vehicle 1, for example.

The following will concretely describe a configuration of the control device 20.

The control device 20 includes a communication unit 210, a control unit 220, and a storage unit 230, as illustrated in FIG. 1.

Communication Unit 210

The communication unit 210 has a function of transmitting and receiving data by wireless or wired communication connection with a device provided in the vehicle 1 (in-vehicle device). The transmission and reception of data with an in-vehicle device may be performed using an in-vehicle communication network conforming to an arbitrary standard such as a controller area network (CAN), a local interconnect network (LIN), or a local area network (LAN), for example. The in-vehicle device is, for example, the above-described locking/unlocking device 30, fragrance generation device 40, odor elimination device 50, and disinfection device 60.

Moreover, the communication unit 210 may have a function of transmitting and receiving data to and from a device existing outside the vehicle 1 (hereinafter, also referred to as an external device). The communication unit 210 may implement a cellular communication protocol such as WiMAX and a long term evolution (LTE), for example, or other wireless communication protocols such as wireless LAN (Wi-Fi) (registered trademark) and Bluetooth (registered trademark). The communication unit 210 may be connected to a server on an external network (the Internet, for example) through a base station or an access point. For example, the communication unit 210 may be communicably connected to the car sharing service server 10 through the network 12.

Control Unit 220

The control unit 220 is a component that collectively controls the entire processing performed by the control device 20. For example, the control unit 220 performs processing for achieving various functions using programs, parameters, and the like stored in the storage unit 230. The control unit 220 may be achieved by a processor such as a central processing unit (CPU) or a micro controller unit (MCU), for example.

Moreover, the control unit 220 of the embodiment also functions as a driving start/end determination unit 221, a fragrance control unit 222, an odor elimination control unit 223, and a disinfection control unit 224.

The driving start/end determination unit 221 has a function of determining a start of driving (a start of use of the vehicle 1) and an end of driving (an end of use of the vehicle 1) that are examples of the use state of the vehicle 1. For example, the driving start/end determination unit 221 may determine a start of driving when the locking/unlocking device 30 has unlocked the door lock of the vehicle 1. Moreover, the driving start/end determination unit 221 may determine a start of driving when the start time for using the vehicle 1 acquired from the car sharing service server 10 (that is, the car sharing booking start time) has come. Moreover, the driving start/end determination unit 221 may determine a start of driving when a use start notification for vehicle 1 has been received from the car sharing service server 10 or a smartphone or the like of a user using the vehicle 1. Moreover, the driving start/end determination unit 221 may determine a start of driving when a use start notification for the vehicle 1 has been received and then the door lock has been unlocked.

Moreover, the driving start/end determination unit 221 may determine an end of driving when the end time for using the vehicle 1 acquired from the car sharing service server 10 (that is, the car sharing booking end time) has come. Moreover, the driving start/end determination unit 221 may determine an end of driving when a use end notification for the vehicle 1 (that is, a car sharing use end notification) has been received from the car sharing service server 10 or a smartphone or the like of a user having used the vehicle 1. Moreover, the driving start/end determination unit 221 may determine an end of driving when a use end notification for the vehicle 1 has been received and then the door lock has been locked.

The fragrance control unit 222 controls the fragrance generation device 40 to provide a fragrance into a vehicle interior (supply a fragrance to target space) when the driving start/end determination unit 221 has determined a start of driving. The configuration of the fragrance generation device 40 is not especially limited. For example, a fragrance is released into the vehicle interior from a fragrance generation unit through a blow-off port. The blow-off system may be a spray type, for example. For example, a fragrance may be liquid including fragrance substances. The fragrance generation unit includes a tank storing a fragrance. Moreover, the fragrance generation unit may include a plurality of tanks storing fragrances. This allows different kinds of fragrances to be released appropriately. The fragrance releasing action may be performed once or a plurality of times, and for an instant or a certain period of time. Moreover, the releasing amount of a fragrance per time may be set appropriately. Moreover, the kind of a fragrance may be in accordance with the preference of a user (a driver), or a fragrance branded for each manufacture or model of the vehicle 1 or for each car sharing service provider. The fragrance control unit 222 can determine a favorite fragrance of a user (a subscriber) on the basis of user information (that is, car sharing booking information) acquired from the car sharing service server 10. For example, a user may select a favorite fragrance when booking car sharing.

Moreover, it is preferable to provide a fragrance immediately before a user (a driver) gets on the vehicle 1 because the fragrance may disappear in a relatively short time. For this reason, in the embodiment, with unlocking of the door lock of the vehicle 1 as a trigger to determine a start of driving, for example, it is possible to release a fragrance immediately before a user gets on the vehicle 1. Note that in order to detect the timing immediately before a user gets on the vehicle 1, the driving start/end determination unit 221 may identify, with a camera (not illustrated) provided in the vehicle 1, for example, a user approaching the vehicle 1 by face recognition or the like.

Moreover, the fragrance control unit 222 may provide a fragrance from the fragrance generation device 40 every time a user gets on the vehicle 1 (that is, every time the door lock is unlocked, for example) while the user uses the vehicle 1. Moreover, it is possible to stop providing a fragrance any time by user's operation or the like on the control device 20.

When the driving start/end determination unit 221 has determined the end of driving, the odor elimination control unit 223 controls the odor elimination device 50 to perform odor elimination in the vehicle interior. The odor elimination device 50 is an example of an odor reduction device that reduces an odor in a vehicle interior. The configuration of the odor reduction device is not especially limited. For example, it is possible to remove an odor by emitting an odor eliminating agent or generating ozone or an ion.

In a vehicle interior, there accumulated odors associated with daily living and food and drink, odors from substances left in the vehicle interior, and the like. Thus, when the end of driving of the vehicle 1 (the end of use of car sharing) has been determined, the odor elimination control unit 223 reduces odors by odor elimination, deodorization, or the like, for car sharing by a next user. In this manner, it is possible to provide comfortable vehicle interior space for a next user. A plurality of odor elimination control units 223 may be provided in the vehicle interior.

When the driving start/end determination unit 221 has determined the end of driving, the disinfection control unit 224 controls the disinfection device 60 to reduce viruses and germs floating or attached in the vehicle interior. The configuration of the disinfection device 60 is not especially limited. For example, it is possible to perform disinfection using ultraviolet light or light with a certain wave length, or by spraying chlorine or the like.

With use of the vehicle 1, germs and viruses may be attached or float in the vehicle interior. Therefore, when the end of driving of the vehicle 1 (end of use of car sharing) has been determined, the disinfection control unit 224 controls the disinfection device 60 to perform disinfection for car sharing by a next user. In this manner, it is possible to provide comfortable vehicle interior space for a next user. A plurality of disinfection control units 224 may be provided in the vehicle interior.

Storage Unit 230

The storage unit 230 is a component that stores various kinds of information. For example, the storage unit 230 stores programs, parameters, and the like used by the control unit 220. Moreover, the storage unit 230 may store processing results by the control unit 220. Note that the contents of information stored by the storage unit 230 are not especially limited. Moreover, the storage unit 230 can be achieved by a read only memory (ROM), a random access memory (RAM), or the like, for example.

The above has described a configuration example of the control device 20 in the embodiment. Note that the configuration of the control device 20 in the embodiment is not limited to the example illustrated in FIG. 1. For example, the control device 20 may be achieved by a plurality of devices. Moreover, the functions of the control unit 220 may be achieved by an electronic control unit (ECU) provided in a vehicle, a microcomputer provided in the ECU, or the like. Moreover, at least a part of the functions of the control unit 220 may be achieved by an external device not provided in a vehicle (a cloud server on the Internet, or a smartphone, tablet terminal, or the like of a driver, for example).

### 2. Action Processing

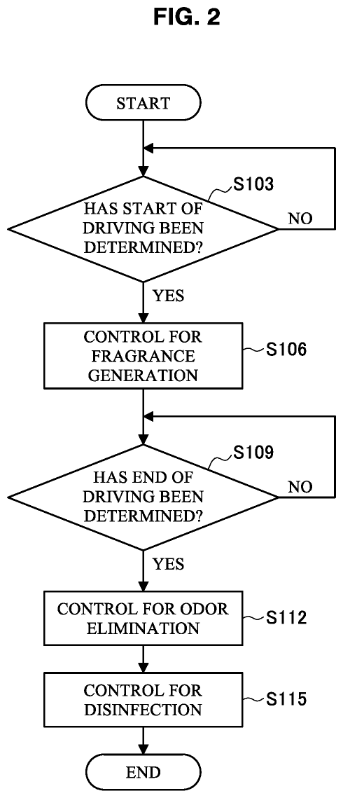

The following will describe the action processing of a space improvement control system according to the embodiment with reference to FIG. 2. FIG. 2 is a flowchart illustrating an example of a flow of a space improvement control according to the embodiment.

As illustrated in FIG. 2, when the driving start/end determination unit 221 has determined a start of driving (Yes at Step S103), the fragrance control unit 222 of the control device 20 controls the fragrance generation device 40 to generate a fragrance (Step S106). The start of driving is determined when the door lock of the vehicle 1 is unlocked or when the use start time based on the use information acquired from the car sharing service server 10 has come, for example.

Next, when the driving start/end determination unit 221 has determined an end of driving (Yes at Step S109), the odor elimination control unit 223 controls the odor elimination device 50 to perform odor elimination (Step S112). The end of driving is determined when a use end notification has been received from the car sharing service server 10 or by user's operation, and the door lock of the vehicle 1 has been locked, for example. Moreover, the odor elimination may be performed for a certain period of time.

Next, the disinfection control unit 224 controls the disinfection device 60 to perform disinfection (Step S115). The disinfection may be performed for a certain period of time.

The above-described processing is repeated continuously, whereby comfortable space is provided also when a plurality of users share the vehicle 1 and use it sequentially. To be more specific, when a user gets on the vehicle 1, the vehicle 1 is disinfected with a reduced odor, whereby the user is able to enjoy a fragrance without being conscious of air cleaning or the like of the vehicle interior. In this manner, even when the vehicle 1 is shared and used with other persons, it is possible to obtain clean feeling and refreshing feeling of the vehicle interior and improve comfortability in vehicle use. Moreover, the disinfection and the like are performed without involving a person, which prevents infectious diseases.

Moreover, although the disinfection is executed after the odor elimination in the above-described example, the embodiment is not limited thereto. The odor elimination may be executed after the disinfection, or they may be alternately executed a plurality of times.

### 3. Supplement

Heretofore, preferred embodiments of the present invention have been described in detail with reference to the appended drawings, but the present invention is not limited thereto. It is obvious that a person skilled in the art can arrive at various alterations and modifications within the scope of the technical ideas defined in the claims, and it should be naturally understood that such alterations and modifications are also encompassed by the technical scope of the present invention.

For example, the space improvement control system of the embodiment is achieved by at least the control device 20, and does not necessarily have a configuration including the car sharing service server 10. Moreover, the control device 20 may acquire the use information of the vehicle 1 from a smartphone or the like of a user using the vehicle 1 by short-distance wireless communication or the like.

Moreover, the space improvement control system of the embodiment may have a configuration including at least any one of the fragrance generation device 40, the odor elimination device 50, and the disinfection device 60 in the vehicle 1. The control device 20 may perform at least any one of generation of a fragrance in accordance with the start of driving, and odor elimination or disinfection in accordance with the end of driving.

Moreover, the determination of an end of driving by the driving start/end determination unit 221 is not limited to the above-described example. For example, the stop of the engine of the vehicle 1 may be an additional condition, or the arrival of the vehicle 1 at a given area such as a parking area may be an additional condition. The given area is an area set as a place to transfer the vehicle 1 to a next user.

Moreover, the above has exemplified a vehicle as a mobile body in which the control device 20 is placed. However, the mobile body of the embodiment is not limited to a vehicle, and may be a ship (a passenger ship, cargo ship, or submarine boat, for example), an aircraft (an airplane, helicopter, glider, or airship, for example), or the like. Moreover, the vehicle is not limited to an automobile, and may be a bus, a motorcycle, a locomotive, a train, or the like. Moreover, the mobile body is not necessarily limited to the above-described examples, and may be any movable object. As an example, the control device 20 is placed in a mobile body, and the control device 20 may be placed at a position other than in a mobile body.

In the above-described embodiment, the vehicle interior of a vehicle is exemplified as target space. However, the embodiment is not limited thereto. For example, the target space may be a guest room of a hotel, a meeting room of a company, or a study room (an individual room), for example.

The effects described in the present specification are merely explanatory or representatives and are not restrictive. That is, the technologies of the present disclosure may exert other effects, in addition to or instead of the above-described effects, that are clear for a person skilled in the art on the basis of the description in the specification.

Moreover, it is also possible to form one or more computer programs controlling a hardware such as a CPU, a ROM, and a RAM in a computer to achieve the functions equivalent to those of the components of the control device 20, and it is also possible to provide a computer readable recording medium recording the one or more computer programs.

What is claimed is:

1. A control device, comprising:
a control unit configured to perform processing of determining a start of use of target space; and
processing of controlling, when the start of use has been determined, a fragrance generation device provided in the target space to supply a fragrance into the target space.

2. The control device according to claim 1, wherein the control device is provided in a vehicle.

3. The control device according to claim 2, wherein the control unit determines a start of driving on the vehicle as the start of use.

4. The control device according to claim 3, wherein the control unit determines the start of driving when a door lock of the vehicle has been unlocked.

5. The control device according to claim 3, wherein the control unit determines the start of driving when car sharing booking time for the vehicle has come.

6. The control device according to claim 2, wherein the control unit performs a control of generating a fragrance in accordance with a preference of a user as a subscriber, on the basis of car sharing booking information of the vehicle.

7. The control device according to claim 2, wherein the control unit performs a control of generating a fragrance branded for each manufacture model of the vehicle or for each car sharing service provider.

8. The control device according to claim 1, wherein the control unit further performs processing of determining an end of use of the target space; and
processing of executing, when the end of use has been detected, an action of at least any one of an odor reduction device and a disinfection device provided in the target space.

9. The control device according to claim 8, wherein the control unit performs a control such that an action of the disinfection device is executed after an action of the odor reduction device is executed.

10. A control device, comprising:
a control unit configured to perform
processing of determining an end of use of target space; and
processing of executing, when the end of use has been detected, at least any one of an odor reduction device and a disinfection device provided in the target space.

11. The control device according to claim 10, wherein the control device is provided in a vehicle.

12. The control device according to claim 11, wherein the control unit determines an end of driving on the vehicle as the end of use.

13. The control device according to claim 12, wherein the control unit determines the end of driving when car sharing booking end time for the vehicle has come.

14. The control device according to claim 12, wherein the control unit determines the end of driving when a car sharing use end notification for the vehicle has been received.

15. The control device according to claim 13, wherein the control unit determines the end of driving when a door lock of the vehicle has been further locked.

16. The control device according to claim 10, wherein the control unit performs a control such that an action of the disinfection device is executed after an action of the odor reduction device is executed.

17. The control device according to claim 10, wherein the control unit further performs
processing of determining a start of use of the target space, and
processing of controlling, when the start of use has been determined, a fragrance generation device provided in the target space to supply a fragrance into the target space.

18. A non-transitory computer readable storage medium storing a program that controls a computer to function as a control unit configured to perform:
processing of determining a start of use of target space; and
processing of controlling, when the start of use has been determined, a fragrance generation device provided in the target space to supply a fragrance into the target space.
